(19) United States
(12) Patent Application Publication
(10) Pub. No.: US 2020/0056220 A1
(43) Pub. Date: Feb. 20, 2020

(54) GENERATION OF DATA FOR USE WITH ANTIMICROBIALS

(71) Applicant: GeneFluidics, Inc., Irwindale, CA (US)

(72) Inventors: Jen-Jr Gau, Pasadena, CA (US); Michael Tomasek, Huntington Beach, CA (US)

(21) Appl. No.: 16/542,699

(22) Filed: Aug. 16, 2019

Related U.S. Application Data
(60) Provisional application No. 62/719,418, filed on Aug. 17, 2018.

(51) Int. Cl. C12Q 1/18; C12N 1/20; G01N 1/34; G01N 1/30; G01N 1/40

(52) U.S. Cl. CPC C12Q 1/18; C12N 1/20; G01N 1/4077; G01N 1/30; G01N 1/34

(57) ABSTRACT

A method of data collection includes culturing a sample that was generated from a raw sample. The raw sample was taken from a patient and the sample includes a pathogen when the raw sample included the pathogen. An assay sample is generated from a portion of the sample such that the assay sample includes the pathogen when the sample included the pathogen. The sample is cultured for a time period that less than hours and greater than or equal to 0.0 hours before the portion of the test sample was taken from the test sample. The assay sample is assayed so as to determine whether the pathogen is present in the assay sample.

### RELATED APPLICATIONS

This Patent Application claims the benefit of U.S. Provisional Patent Application Ser. No. 62/719,418, filed on Aug. 17, 2018, entitled “Generation of Data for Use with Antimicrobials,” and incorporated herein in its entirety. This Patent Application is related to U.S. patent application Ser. No. 12/582,725, filed on Apr. 30, 2017, entitled “Generation of Data for Use with Antimicrobials” and incorporated herein in its entirety.

### FIELD

The invention relates to generation of data and more particularly to use of data related to pathogens and/or associated antimicrobials.

### BACKGROUND

Antimicrobials such as antibiotics are generally prescribed without the physician knowing the identity of the pathogen being treated or if a pathogen is even present. This practice has resulted in the overprescription of antibiotics that has led to antibiotic-resistant pathogens. This practice has likely developed as a result the long time (˜48-72 hours) required for a physician to get lab results that indicate the presence and/or identity of pathogens when the physician is facing patients that want rapid treatments. This practice may also have developed due to the costs of identifying the pathogen exceeding the cost of treating with antimicrobials.

Another source of drug resistant pathogens is prescribing the incorrect dosage of antimicrobials to patients. For instance, the pathogen can develop resistance to the antimicrobial when the dosage is undesirably low. Undesirably high dosages of an antimicrobial can be harmful to organs such as the liver.

For the above reasons, there is a need for improvements in pathogen identification and the use of antimicrobial data.

### SUMMARY

A method of data collection includes viability culturing a test sample that was generated from a raw sample. The raw sample can be taken from a patient or other raw source of pathogens. The test sample includes a pathogen when the raw sample included the pathogen. An assay sample is sample is generated from a portion of the test sample such that the assay sample includes the pathogen when the test sample included the pathogen. The test sample is cultured for a period of time that is less than 8 hours and greater than or equal to 0 hours before the portion of the test sample was taken from the test sample. The assay sample is assayed so as to determine whether the pathogen is present in the assay sample. The assay can be the first assay that is performed on a sample that is generated from all or a portion of the raw sample and indicates whether the pathogen is present in the raw sample.

Another embodiment of a method of data collection includes culturing a viability sample that was generated from a raw sample. The method also includes assaying an assay sample for the presence or absence of a pathogen in the raw sample. The method further includes testing an AST test sample for growth of the pathogen in the presence of an antimicrobial. The AST sample and the assay sample are each generated from the viability sample.

### BRIEF DESCRIPTION OF THE FIGURES

FIG. 1 a process flow for a pathogen characterization phase.

FIG. 2 is an example of a process flow of an antimicrobial susceptibility testing phase.

### DESCRIPTION

The inventors have developed electrochemical sensors that can be used to assay samples for the presence and/or concentration of pathogens. Each assay is typically specific to a particular pathogen. As a result, when multiple pathogens are possible, multiple different assays can be performed on a sensor array with each of the assays being associated with one of the possible pathogens. If any of the assays indicates the presence of the pathogen, then pathogen(s) associated with those assays is/are present. As a result, the assays can identify the pathogen.

These assays have proven to have very low Limits Of Detection (LOD). For instance, in urine samples, these assays can have an LOD of 75,000 CFU/mL (Colony Forming Units/mL) without the need of the initial culturing or enrichment to increase pathogen concentration. After 30 and 90 minutes of performing a fully automated built-in viability culture on the sample, the LOD of same assay drops to 30,000 CFU/mL and 10,000 CFU/mL respectively. In whole blood, these assays can have a LOD of 2,000 (CFU/mL) without culturing to increase pathogen concentration. After 240 and 300 minutes of performing a fully automated built-in viability culture on the whole blood sample, the LOD of the same assay drops to less than or equal to 35 CFU/mL and 1.0 CFU/mL respectively. As a result, little or no culturing time is needed in order to achieve accurate assay results even at low LOD levels. Further, these assays are easily performed in the same facilities where samples are taken from patients. Accordingly, the time associated with transporting samples to off-site facilities is also eliminated. Further, the amount of assay time needed to perform one of these assays on a sample is generally on the order of 30-120 minutes from the time the person(s) performing the assay receives the sample. For instance, when a viability culture is performed, the assay time needed to complete the assay after passage of a viability culture time is between 30-120 minutes. When a viability culture is not performed, the assay time needed to complete the assay after the viability culture is between 30-120 minutes after person(s) performing the assay receives the test sample or the raw sample that will serve as the test sample. Accordingly, it is possible to quickly receive the results of assays that are accurate at low LOD levels.

The samples used in these assays (assay samples) can be prepared using an enrichment step that increases the concentration of the pathogen in the sample to achieve a lower LOD and/or removes matrix interfering components from raw specimens. The enrichment step can be repeated one or more times. If the pathogen is present, the concentration of pathogen in the test sample increases with increasing number of enrichment steps. As a result, the assay becomes more likely to detect the pathogen as the pathogen concentration increases and matrix interfering components decreases. Accordingly, the test sample can be repeatedly concentrated for better detection of the pathogen directly from the raw specimen. However, such an approach can require additional equipment and assay supplies, as well as continual use of manpower and assay equipment. A robotic system such as the UtiMax or BsiMax from GeneFluidics, Inc. of Irwindale, Calif., USA allows the same or even better results to be achieved without a separate culture system, a concentration system, a purification system or repeated assaying. As a result, it is possible to quickly and accurately identify if a pathogen is present, the identity of pathogen(s) that are present, and the susceptibility of pathogen(s) to pre-selected antimicrobials with practical levels of expense, assay time and manpower.

This ability to quickly characterize the pathogen can revolutionize treatment with antimicrobials such as antibiotics such as Cefepime, and Meropenem. For instance, knowing that a pathogen is not present can prevent the prescription of antimicrobials in situations when they are not effective. Additionally, knowing the identity of pathogen(s) that are present allows for the prescription of antimicrobials that are known to be effective against the pathogen(s). Further, the quick availability of these results permits these prescriptions to be made in a time period that is convenient for the patient. For instance, these prescriptions can often be made in several hours. As a result, these prescriptions can generally be made on the same day a sample is taken from the patient, while the patient is on the way home from the site where the sample was taken, or while the patient is still at the site where the sample was taken. Since antimicrobials will be tailored to the pathogens that are present, proliferation of antimicrobial-resistant pathogens can be reduced.

The ability to quickly know whether a pathogen is present in a sample can further permit screening of antimicrobials before a prescription of an antimicrobial is made. For instance, the sample taken from a patient can be cultured in the presence of the antimicrobial(s) that are selected as being effective against the identified pathogen(s). In some instances, these susceptibility cultures are performed using different concentrations of the identified antimicrobial(s). The same assay technologies can be used to measure growth of the pathogen in these susceptibility cultures. These results can be compared to identify the antimicrobial's susceptibility, and, in some instances, Minimum Inhibitory antimicrobial Concentrations (MICs) of the identified pathogens. The antimicrobial to which the pathogen is most susceptible can then be prescribed. Additionally or alternately, the dosage of the prescription can be based on the Minimum Inhibitory antimicrobial Concentration (MICs). For instance, when lower concentrations of an antimicrobial are shown to be effective against a pathogen, lower doses of the antimicrobial can be prescribed. When higher concentrations of an antimicrobial are shown to be effective against a pathogen, the prescribed dosage of the antimicrobial can be increased. As a result, the initial prescription given to a patient can be experimentally shown to be effective against the strain of pathogen identified in raw sample taken from a patient. The use of experimental results to tailor the prescription of antimicrobials to the identified pathogen would reduce the use of broad-spectrum antimicrobials and can lead to a subsequent drop in the proliferation of antimicrobial-resistant pathogens.

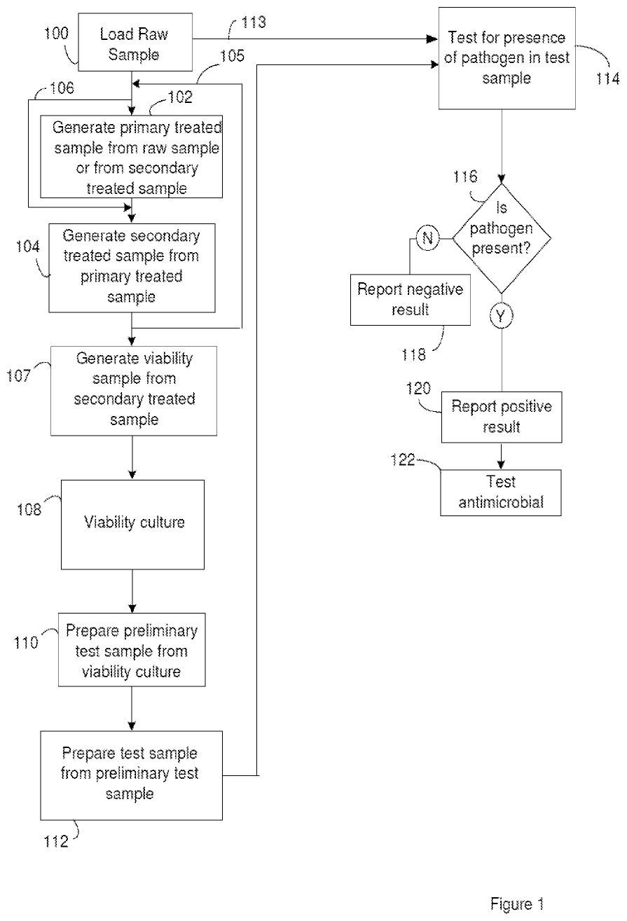

The assays described above can be used in a pathogen characterization phase where the presence or absence of a pathogen in a raw sample is determined, any pathogen(s) in the sample are identified, and/or a concentration of any pathogen(s) in the sample is determined. The inventors have found a method of preparing raw samples before performing the assay(s) that provides the assays with a very low Limits Of Detection (LOD) in very short times after taking the raw sample. FIG. 1 is a flow diagram illustrating this method incorporated into a pathogen characterization phase. The method can be fully or partially performed by a robotic system such as the UtiMax or BsiMaxUtiMax or BsiMax from GeneFluidics, Inc. of Irwindale, Calif., USA. When such a system is used, a user receives a raw sample. At process block 100, the user can load the raw sample into the system. The raw sample can be taken from a raw source of pathogens such as food, the environment, or a biological source. Alternately, the raw sample can be taken from a patient such as a person or animal that is living or dead. Examples of raw samples taken from a patient include, but are not limited to, whole blood, urine, nasal swab, saliva, and cerebrospinal fluid (CSF). The raw sample can be in a container such as a sample tube. The system can read an indicator on the container such as a sample tube to identify one or more features about the raw sample. For instance, the system can read a bar-code on the container. The bar code can identify the type of raw sample to the system. When the raw sample is taken from a source other than a patient, the type of raw source can be identified by the identifier on the container or entered by a user using a graphic user interface (GUI) on the system. The system can identify the one or more assays that are associated with the identified raw sample. The system can execute the remainder of the FIG. 1 flow diagram so as to perform the one or more assays on the raw sample.

At process block 102, a primary treated sample is generated from all or a portion of the raw sample. The primary treated sample is generated such that the primary treated sample includes a target pathogen when the raw sample included the target pathogen. Generating the primary treated sample can include performing an optional purifying operation on a sample that includes or consists of all or a portion of the raw sample. The purifying operation can reduce the number and/or concentration of at least one type of cells other than the one or more pathogens that are the target of the identified assays (target pathogens). For instance, when the raw sample includes cells in addition to the target pathogens, the purifying operation can be performed so as to reduce the concentration and/or number of the non-pathogen. For instance, all or a portion of the raw sample can be lysed so as to reduce the number and/or concentration of non-target pathogens in the sample that includes or consists of all or a portion of the raw sample. As an example, raw samples of whole blood include red blood cells (RBCs) that are not target pathogens. Accordingly, an RBC lysing agent can be used on the raw sample to selectively lyse RBC, but not target pathogens. Suitable RBC lying reagents include, are but not limited to, 1X RBC Lysis Buffer (ThermoFisher cat. no. 00-4333), ACK Lysing Buffer (ThermoFisher cat. no. A1049201), and Red Blood Cell Lysis Buffer (Roche 11814389001)

The medium upon which the purifying operation was performed can serve as the primary treated sample. For instance, when the purifying operation is performed on all or a portion of a raw sample and one or more of the target pathogens were present in the raw sample, the resulting medium includes the pathogen and can serve as the primary treated sample. As an example, when all or a portion of a raw sample is lysed, the lysate can serve as the primary treated sample. In some instances, the raw sample will not include any of the target pathogen.

At process block 104, a secondary treated sample is generated from all or a portion of the primary treated sample. The secondary treated sample is generated such that the secondary treated sample includes a target pathogen when the primary treated sample included the target pathogen and accordingly when the raw sample included the target pathogen. Generating the secondary treated sample can include performing a pathogen concentration operation on all or a portion of the primary treated sample (or the raw sample). When the raw sample includes the target pathogen(s), the pathogen concentration operation can provide a secondary treated sample that has the target pathogen at a higher concentration than the pathogen concentration in the primary treated sample.

Suitable methods for performing the pathogen concentration operation include, but are not limited to, filtering, sedimentation and/or centrifugal separation techniques such as spinning, centrifuging, pipetting, supernatant removal, and cellular pelleting. Centrifuging is an effective approach for concentrating pathogens such as bacteria because bacteria are heavier and bigger than hemoglobin, lipids, and other molecules found in the matrices of clinical specimens. Accordingly, centrifuging of the enriched sample causes pathogens such as bacteria to concentrate in the centrifuge pellet rather than the supernatant. As a result, the supernatant can be separated from the centrifuge pellet and the centrifuge pellet can serve as the secondary treated sample.

The generation of the primary treated sample and the secondary treated sample can serve as a step in an enrichment cycle that can be repeated one or more times as illustrated by the arrow labeled 105. When the enrichment cycle is repeated more than once, the primary treated sample can be generated from the prior secondary treated sample after the first cycle. As a result, generation of the primary treated sample and the secondary treated sample can be repeated multiple times. The repeated generation of the primary treated sample and the secondary treated sample can provide a final secondary treated sample with a concentrated level of the target pathogen(s) and/or reduced concentrations of non-target pathogen. In some instances, the cycle is repeated more than once, twice, or three times and/or less than eight, ten, or a hundred times.

As illustrated by the arrow labeled 106, generating the primary treated sample is optional. When the primary treated sample is not generated, the secondary treated sample can be generated from all or a portion of the raw sample. Accordingly, the raw sample can serve as the primary treated sample when the primary treated sample is not generated from the raw sample.

At process block 107, a viability sample is generated from the secondary treated sample. The viability sample is generated such that the viability sample includes a target pathogen when the secondary treated sample included the target pathogen and accordingly when the raw sample included the target pathogen. Generating the viability sample can include combining all or a portion of the secondary treated sample with one or more culture media. At process block 108, the viability sample is cultured so as to increase the concentration of the one or more target pathogens in the viability sample. The culturing of the viability sample is performed for a viability culture time. In some instances, the viability culture time is a function of the desired LOD level or a clinical cutoff.

At process block 110, after the viability sample has been cultured for the viability culture time, a preliminary test sample is generated from an aliquoted portion of the viability sample and accordingly from all or a portion of the viability sample. The preliminary test sample is generated such that the preliminary test sample includes a target pathogen when the viability sample included the target pathogen and accordingly when the raw sample included the target pathogen. Generating the preliminary test sample can include aliquoting a portion of the viability sample from the viability sample. The aliquoted portion of the viability sample can be the entire viability sample or a fraction of the viability sample.

Generating the preliminary test sample can include performing a pathogen concentration on the aliquoted portion of the viability sample. The pathogen concentration provides a preliminary test sample having any pathogen at a higher concentration than the pathogen concentration in the viability sample. Suitable methods for concentrating the viability sample include, but are not limited to, filtering, sedimentation and/or centrifugal separation techniques such as spinning, centrifuging, pipetting, supernatant removal, and cellular pelleting. Centrifuging is an effective approach for concentrating pathogens such as bacteria because bacteria are typically denser than hemoglobin, lipids, and other molecules found in the matrices of clinical specimens. Accordingly, pathogens such as bacteria concentrate in the centrifuge pellet rather than the supernatant. Accordingly, the supernatant can be separated from the centrifuge pellet and the centrifuge pellet can serve as a preliminary test sample.

At process block 112, a test sample is generated from all or a portion of the preliminary test sample. The test sample is generated such that the test sample includes a target pathogen when the preliminary test sample included the target pathogen and accordingly when the raw sample included the target pathogen. Generating the test sample can include performing a release operation on a sample that includes or consists of all or a portion of the preliminary test sample. The release operation can release RNA content from at least a portion any of the suspected pathogen(s) in the preliminary test sample. For instance, when the preliminary test sample includes non-target cells and one or more of the suspected pathogens can be found within the raw sample, all or a portion of the preliminary test sample can be lysed so to release the RNA content from target pathogens. As an example, when the preliminary test sample is generated from a whole blood raw sample that is to be assayed for a particular bacterium, all or a portion of the resulting preliminary test sample can be lysed so as to release the RNA content from suspected bacteria. The lysing agent can be a function of the suspected pathogen. For instance, a Gram-positive lysing agent can be used when a suspected pathogen is a Gram-positive bacteria and an Gram-negative lysing agent can be used when a suspected pathogen is a Gram-negative bacteria. Other suitable methods for releasing the one or more suspected pathogens from cells include, but are not limited to, chemical lysing, mechanical lysing, and electrical lysing.

In addition to the release operation or as an alternative to the release operation, generating the test sample can include a dividing operation. The dividing operation can include aliquoting or dividing all or a portion of the preliminary test sample or the released preliminary test sample into at least two portions. Performing the aliquoting or dividing operation can generate at least two test samples. For instance, a portion of preliminary test sample or released preliminary test sample can be removed and placed in a separate container. The remaining portion of the preliminary test sample in the original container and the preliminary viability sample in a second container will share the same characteristics including target pathogen(s) and concentration(s) of target pathogen(s) when present in the preliminary test sample.

When a release operation and/or a dividing operation is performed on all or a portion of the preliminary test sample, the result can serve as the test sample. For instance, when a preliminary test samples sample is lysed, the lysate can serve as the test sample. Alternately, when a dividing operation is performed on the preliminary test sample, one or more of the portions of the preliminary test sample can serve as the test sample. When the dividing operation and the release operation are performed on the preliminary test sample, the dividing operation can be performed before or after the release operation. When the dividing operation is performed before the release operation, a portion of the preliminary test sample upon which the release operation was not performed can serve as one or more of the test samples and another portion of the preliminary test sample upon which the release operation was performed can serve as one or more of the test samples. For instance, when the dividing operation is performed before the release operation, a portion of the preliminary test sample upon which the release operation has not been performed can serve as one or more of the test samples and another portion of the preliminary test sample upon which the release operation has been performed can serve as one or more of the test samples.

As is illustrated by the arrow labeled 113 in FIG. 1, in some instances, the raw sample can serve as the test sample. For instance, in some instances where the raw sample is urine samples, culture media, spiked sample in buffer, or purified sample, it is not necessary to provide the sample preparation and culturing associated with process block 102 through process block 112. When direct-raw sample-to-testing is to be used for a raw sample, the identifier on the raw sample container can indicate this to the system.

At process block 114, one of the one or more test samples is tested so as to generate results that include one or more factors selected from the group consisting of whether a pathogen is present in the raw sample, the identity of a pathogen in the sample, and the level and/or concentration of the pathogen in the sample over pre-determined cutoff. When one or more test samples are available upon which a release operation has been performed and one or more test samples are also available upon which a release operation has not been performed, the test can be performed on one of the test samples for which the release operation has been performed.

The test of a test sample can include one or more assays that are each specific to a particular pathogen and/or group of pathogens. As a result, an assay that is specific to a particular pathogen indicating that a pathogen is present in the test sample also identifies the pathogen and/or a pathogen group. When the assay is specific to a particular pathogen and multiple different pathogens are suspected to be present in the raw sample, multiple assays can be performed on the test sample where at least a portion of the assays are directed to different target pathogens or pathogen groups.

When the assays are to be performed using an electrochemical sensor, all or a portion of the test sample can be suitable for use with the electrochemical sensor as an assay sample and/or the test sample can be combined with one or more reagents to form an assay sample that is suitable for use with the electrochemical sensor. Accordingly, the assay sample can include, consist of, or consist essentially of the test sample. The assay sample can be in direct physical contact with the electrochemical sensor and/or can be in direct physical contact with an electrode on the electrochemical sensor. In one example, the assay sample is a drop of liquid in contact with a working electrode on the electrochemical sensor.

In the above description, the viability culture time is the minimum period of time over which the test sample is cultured before preparing the preliminary test sample or before preparing the test sample in the case where a preliminary test sample is not generated. For instance, a viability culture time of 3 hours indicates that the viability sample has been cultured for at least 3 hours before the aliquoted portion of the viability sample is taken. Accordingly, the preliminary test sample and the test sample are generated after at least the viability culture time period has passed.

Using the above method of preparing the viability sample has provided very low Limits Of Detection (LOD) even when using very low culture times. For instance, for raw samples of whole blood, Limits Of Detection (LOD) less than 100, or 10 CF/mL have been achieved when using culture times less than 2 hours, or 3 hours. For raw samples of urine, Limits Of Detection (LOD) less than 10,000 CFU/mL have been achieved when using culture times less than 1.5 hours and LOD less than 3E4 CFU/mL have been achieved when using culture times less than 30 minutes. For raw samples of culture media, LOD less than 10,000 CFU/mL have been achieved when using culture times less than 30 minutes and LOD less than 50,000 CFU/mL have been achieved without any viability culture.

The inventors have found that pathogen concentration in raw samples is typically at the lower end of the possible concentration range. For instance, pathogen concentration in raw samples is typically on the order of <10 CFU/mL for blood, >10,000 CFU/mL for urine, <2 CFU/mL for lake water, <100,000 CFU/gram for cooked meat. The viability culture time can be selected to provide a Limit Of Detection (LOD) that is less than these concentration levels to ensure that any of the target pathogen in the raw sample is detected. Additionally, the viability culture time can be a function of the raw sample. For instance, for raw samples of whole blood, the viability culture time can be less than 6 hours, 4 hours, or 3 hours and greater than 0 hour, 1 hours, or 2 hours. For raw samples of urine, the viability culture time can be less than 4 hours, 3 hours, or 2 hours and greater than 0 hour, 30 minutes, or 60 minutes. For raw samples of culture media, the viability culture time can be less than 4 hours, 3 hours, or 2 hours and greater than 0 hour, 30 minutes, or one hour. Accordingly, suitable viability culture times include times less than 8 hours, 6 hours, or 3 hours, and/or greater than or equal to 0.0 hours, or 30 minutes.

The above viability culture times can reduce the total assay time. A total assay time can be the period of time that lapses between taking and/or receiving the raw sample and characterizing the target pathogen. When a robotic system is employed to do the viability culture and the corresponding assay(s), the total assay time can be the period of time that lapses after loading the raw sample into the system. Accordingly, when a viability culture is performed, the total assay time can include the time to generate the viability sample from the raw sample, the viability culture time and the assay time to perform the assays on the test sample. When a viability culture is performed, the total assay time can include the time to generate the test sample from the raw sample and the assay time to perform the assays on the test sample. The pathogen characterization can include the determination of one or more factors selected from the group consisting of whether one or more of the suspected pathogens is present in the raw sample and identifying one or more pathogens present in the raw sample. Suitable total assay times include, but are not limited to, times less than 8 hours, 6 hours, 2 hours, or 30 minutes. As noted above, in some instances, the raw sample serves as the test sample and culturing is not required. In these instances, the total assay time can be substantially reduced. For instance, the total assay time can be less than 30 minutes, one hour, or two hours.

Because the Limit Of Detection (LOD) for the above assay(s) is less than the pathogen concentration levels in raw samples, the assay(s) described in the context of FIG. 1 can be the first assays performed on the raw sample. For instance, the pathogen characterization phase can be conducted such that before the aliquoted portion of the viability sample is taken, no other portion of the viability sample is taken from the viability sample and subsequently assayed so as to determine whether a pathogen is present in the raw sample.

Since the method of preparing the assay sample brings the LOD below the pathogen concentration seen in raw samples, additional culturing of the viability sample may not be needed after the aliquoted portion of the viability sample is taken from the viability sample. Accordingly, in some instances, culturing of the viability sample is stopped after the aliquoted portion is taken from the viability sample. Alternately, culturing can be continued so a source of viability sample remains available in the case of errors in subsequent treatment of the aliquoted portion of the viability sample.

At decision block 116, a determination is made whether one or more of the target pathogens is present in the test sample. If the determination is negative, a negative result is reported at process block 118 indicating that the one or more pathogens associated with the assay(s) used during the testing are not present in the raw sample. If the determination is positive, a positive result is reported at process block 120 indicating that one or more pathogens associated with the assay(s) used during the testing are present in the raw sample. At process block 122, an antimicrobial susceptibility testing phase can optionally be started as is described in more detail below.

The assays in the above flow can be performed manually. Alternately, as disclosed above, the assays in the above flow can be performed using a robotic system such as the UtiMax or BsiMaxUtiMax or BsiMax from GeneFluidics, Inc. of Irwindale, Calif., USA. A suitable electrochemical sensor for use in performing the above assays is the UtiMax sensor chip and/or the BsiMax ID/AST sensor chip sold by GeneFluidics, Inc. of Irwindale, Calif., USA. The assays can be performed by using these electrochemical sensors manually or with one of the robotics systems such as the UtiMax or BsiMaxUtiMax or BsiMax Robotic System and/or the Lab Automation System sold by GeneFluidics, Inc. of Irwindale, Calif., USA. The reagents used in these assays can be purchased in reagent kits. For instance, a reagent kit for use with urinary tract infections is the UtiMax sold by GeneFluidics, Inc. of Irwindale, Calif., USA. Pathogens that can be identified with the UtiMax reagent kit include E. coli, P. aeruginosa, and K. Pneumoniae and more depending on the ID sensor chip configuration. The Limit of Detection (LOD) for an assay performed using the UtiMax reagent kit in combination with the with the UtiMax or BsiMax system and the above method of preparing the assay sample has been shown to be 10,000 CFU/mL with zero culture time before taking the aliquoted portion of the viability sample. A reagent kit for use with bloodstream infections is the BsiMax sold by GeneFluidics, Inc. of Irwindale, Calif. Pathogens that can be identified with the BsiMax reagent kit include E. coli, P. aeruginosa, and S. aureus and more depending on the ID sensor chip configuration. The Limit of Detection (LOD) for an assay performed using the BsiMax reagent kit in combination with the UtiMax or BsiMax system and the above method of preparing the assay sample has been shown to be 4 (CFU/mL) with an 5-hour culture time before taking the aliquoted portion of the viability sample. These LOD numbers are determined according to Clinical and Laboratory Standards Institute (CLSI)I document EP17-A, “Evaluation of Detection Capability for Clinical Laboratory Measurement Procedures.” The UtiMax and the BsiMax reagent kits come with electrochemical sensors that can be used manually or with a robotic system such as the UtiMax or BsiMax from GeneFluidics, Inc. of Irwindale, Calif., USA.

The assay sample can be prepared manually from the raw sample and/or the assay sample can be manually assayed. Alternately, as disclosed above, a robotic system such as the UtiMax or BsiMax can be used to prepare the assay sample from the raw sample and/or to perform the assay of the assay sample. When a robotic system such as the Proteus prepares the assay sample, the system can automatically perform the entire assay from receiving the raw sample, through preparing the assay sample to testing of the assay sample. When the assay sample is prepared and the assay is performed, the UtiMax or BsiMax robotic system accesses vials containing reagents for preparing the assay sample from the raw sample from the UtiMax or BsiMax reagent kits located in designated reagent rack(s). For instance, the reagents can include lysing solutions, buffers, enzyme, AST medium, and TMB. The UtiMax or BsiMax robotic system includes a centrifuge for performing concentration operations on one or more samples prepared by the UtiMax or BsiMax robotic system. The UtiMax or BsiMax robotic system can transfer the lysate and the needed reagents onto the electrochemical sensors. In some instances, the UtiMax or BsiMax robotic system also adds one or more reagents to the test sample in order to form the assay sample. When the UtiMax or BsiMax robotic system does not add any more reagents to the test sample, the test sample serves as the assay sample. The UtiMax or BsiMax robotic system can incubate the test sample on the sensor followed by one or more optional operations such as washing, drying, and enzyme incubation. The UtiMax or BsiMax robotic system can perform electrochemical reading by adding TMB substrate onto each sensor and operating a multi-channel potentistat connected to the sensors. For instance, the UtiMax or BsiMax robotic system performs amperometry or cyclic voltammetry on the sensors. The UtiMax or BsiMax robotic system provides the results to a user. For instance, the UtiMax or BsiMax robotic system can indicate to a user the pathogen species identified by the system based on the sensor chip used for testing. It can also indicate if multiple pathogens have been detected and, can often identify each of the species detected. Additionally, if the pathogen species is not identified by one of the species-specific sensors, it can indicate whether the tested sample contains a Gram-negative or Gram-positive sample. The UtiMax or BsiMax will also indicate whether the universal sensor that will detect any bacteria has produced a signal.

When using a robotic system such as the UtiMax or BsiMax to perform an assay, the testing time period from a user receiving the raw sample to a user receiving the results of the assay (an indication or positive or negative and/or concentration of pathogen) is generally on the order of 30-360 minutes. When manually performing an assay, the testing time period from a user receiving the raw sample to a user receiving the results of the assay (an indication or positive or negative and/or concentration of pathogen) is generally on the order of 30-360 minutes. Accordingly, the assays described above can be performed in a testing time period greater than 30 minutes, 60 minutes, or 120 minutes and/or less than 4 hours, 5 hours, or 6 hours.

The preparation of the assay sample from the raw sample and the subsequent testing can be performed on site. For instance, since the testing can be performed using robotic systems such as UtiMax or BsiMax, the testing can occur in the same room, building, or medical complex where the raw sample was taken and/or where the assay sample was prepared from the raw sample. As a result, the time delay associated with transportation of the raw sample to an off-site location can be removed. Further, the total time to generate results from the pathogen identification phase are the cumulative times of the time to prepare the viability sample from the raw sample, the viability culture time, the time to prepare the assay sample from the aliquoted portion of the viability sample, and the time to test the assay sample. Once a raw sample becomes available, the viability sample can generally be generated in times less than 360 minutes, 180 minutes, or 30 minutes and/or greater than 0 minute, 30 minutes, or 180 minutes inside UtiMax or BsiMax. The assay sample can generally be prepared from an aliquoted portion of a viability sample in times less than 5 minutes, 2 minutes, or 1 minute and/or greater than 5 seconds, 15 seconds, or 1 minute. Accordingly, the time period between taking the raw sample and completing the pathogen characterization can be less than 120 minutes, 270 minutes, or 450 minutes after taking the raw sample. Accordingly, it becomes possible to receive the results of the pathogen identification phase in a time period less than 30 minutes, 180 minutes, or 360 minutes after taking the raw sample.

In view of the above results, the pathogen identity and/or concentration can be taken into account when prescribing an antimicrobial in times as little as 120 minutes and extending up to 270 minutes, 330 minutes, or 450 minutes. The prescription can be based on guidelines that associate one or more particular antimicrobial medications with particular pathogens. Accordingly, the pathogen identity(ies) can be compared to the prescription guidelines to identify one or more antimicrobials. The comparison of the pathogen identity(ies) to the guidelines can be done manually or can be done by electronically. For instance, the guidelines can be programmed into a robotic system such as the UtiMax or BsiMax and the robotic system can then communicate the one or more suggested antimicrobials to a user.

In some instances, the raw sample can enter an antimicrobial susceptibility testing phase after the pathogen characterization phase. In some instances, the antimicrobial susceptibility testing phase is performed before an antimicrobial is prescribed and the results of the antimicrobial susceptibility testing phase are taken into account when prescribing the antimicrobial. In an antimicrobial susceptibility testing phase, the susceptibility of the identified pathogen(s) to an identified antimicrobial is tested. For instance, an antibiogram can be established at each hospital. The antibiogram can be taken into account when making the prescription. For instance, a physician, pharmacist, user, technician, or other person authorized to write prescriptions can prescribe the antimicrobial to which the identified pathogen(s) is most susceptible or can refrain from prescribing any antimicrobials when the pathogen(s) are not susceptible to any of the identified antimicrobials.

The low Limits Of Detection (LOD) associated with the pathogen characterization phase described above also permits the antimicrobial susceptibility testing phase to be performed in a period of time that allows a prescription to be made that is both in a reasonable period of time after taking the raw sample and is based on antimicrobial susceptibility information. For instance, the raw sample can be cultured in the presence of each one of the antimicrobials that are identified as described above. A different culture can be performed for each of the identified antimicrobials. Additionally, one or more cultures can be performed for each of the identified antimicrobials. When more than one culture is performed for a single antimicrobial, the different cultures can be performed with different concentrations of antimicrobial.

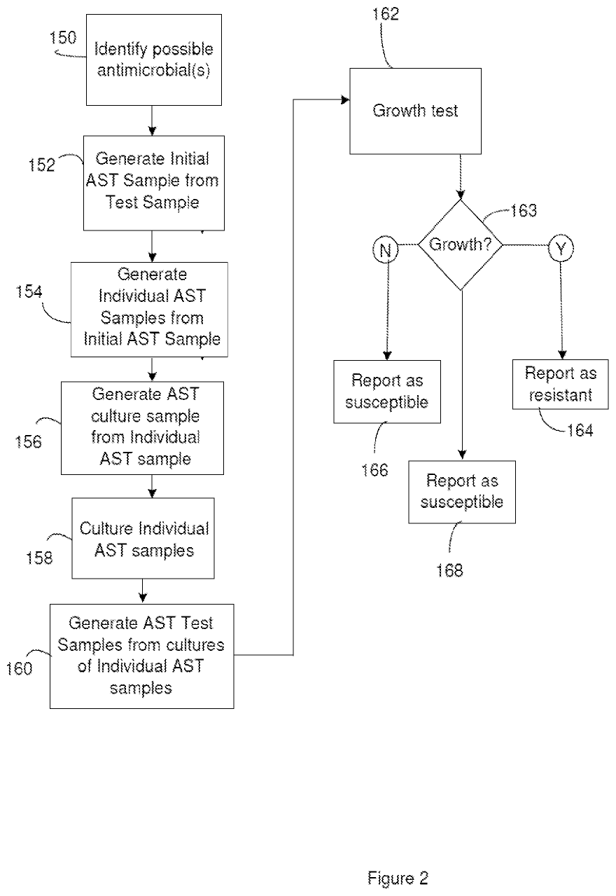

FIG. 2 presents a process flow for an example of an antimicrobial susceptibility testing phase. At process block 150, one or more antimicrobials that are possible candidates for treating the one or more pathogens identified in the pathogen characterization phase. The one or more antimicrobials can be identified as described above. For instance, the one or more antimicrobials can be identified using guidelines that associate one or more particular antimicrobials with particular pathogens.

At process block 152, one or more initial Antimicrobial Susceptibility Testing samples (initial AST samples) are generated. These samples can be generated from one of the test samples generated as disclosed in the context of process block 112 of FIG. 1. When one or more test samples are available upon which a release operation has been performed and one or more test samples are also available upon which a release operation has not been performed, the initial AST sample can be generated from one or more of the test samples upon which a release operation has not been performed. In some instances, one or more test samples are available upon which a release operation has been performed and one or more test samples are also available upon which a release operation has not been performed, the assay sample is generated from one or more of the test samples upon which the release operation has been performed and the initial AST sample is generated from one or more of the test samples upon which the release operation was not performed. In some instances, one or more test samples are available upon which a release operation has been performed and one or more test samples are also available upon which a release operation has not been performed, the assay sample is generated from one or more of the test samples upon which the release operation was been performed but not generated from any test sample upon which the release operation was not performed and the initial AST sample is generated from one or more of the test samples upon which the release operation was not performed but not from any of the test samples upon which the release operation was performed.

In some instances of generating the AST samples, one or more of the test samples each serves as all or a portion of one of the initial AST samples. Additionally or alternately, the initial AST samples can each include or consist of a portion of one of the test samples. For instance, one or more of the initial AST samples can be an aliquot of one of the test samples. In some instances, the number of initial AST samples is at least equal to the number of antimicrobials that are identified in process block 150 plus one (growth control).

In some instances, generating the initial AST sample includes diluting one or more test samples that will be included in the one or more initial AST sample(s). Suitable reasons for this dilution operation include, but are not limited to, high starting concentration of target pathogen in the raw sample, overgrowth during the pathogen identification phase, and predicated concentration over 5E5 CFU/mL. Suitable diluents for the dilution operation include, but are not limited to, Mueller-Hinton broth, Tryptic Soy Broth, and Brain-Heart Infusion broth.

At process block 154 individual Antimicrobial Susceptibility Testing samples (individual AST samples) are generated from the initial AST samples. Generating at least a portion of the individual AST samples can include combining all or a portion of an initial AST sample with one or more of the identified antimicrobials. Different individual AST samples can be generated such that they include the same antimicrobial but at different concentrations. Accordingly, the effectiveness of different concentration level of the identified antimicrobials can be explored. The individual AST samples can be generated such that one or more of the individual AST samples excludes any of the identified antimicrobials. The one or more of the individual AST samples that exclude the identified antimicrobials can serve as a control AST samples (growth control). Accordingly, each of the individual AST samples can include all or a portion of each of the initial AST samples.

At process block 156, Antimicrobial Susceptibility Testing culture samples (AST culture samples) are generated from the individual AST samples. Generating the AST culture samples can include combining all or a portion of each individual AST sample with one or more culture media. Accordingly, each of the AST culture samples can include all or a portion of one of the individual AST samples.

At process block 158, the AST culture samples are cultured such that the concentration of the pathogen in the AST culture samples would increase in the absence of the identified antimicrobials. The culturing of the AST culture samples is performed for an AST culture time. The AST culture time can be greater than 30 minutes, 90 minutes, or 120 minutes and/or less than 4 hours, 2 hours, or 1 hour. In some instances, the AST culture time is a function of the pathogen identified, antimicrobial to be tested, and/or starting concentration of the initial AST sample. As noted above, more than one AST culture sample can include the same antimicrobial at different concentrations. As a result, each of the different cultures can be performed on an AST culture sample having a different concentration of the antimicrobial. Accordingly, the susceptibility of a pathogen to different concentrations of an antimicrobial can be tested.

At process block 160, AST test samples are optionally generated from the AST culture samples. In some instances, the AST test samples are generated by adding lysing reagents to lyse target pathogens. The one or more AST test samples generated from the control AST samples serve as control test samples.

At process block 162, the AST test samples are tested so as to generate results that indicate whether there has been growth of the one or more identified pathogens in each of the AST test samples including the one or more control test samples. When AST test samples are not generated from the AST culture samples, the AST culture samples serve as the AST test samples. In some instances, one or more growth indication factors are used to test growth of the one or more identified pathogens. The one or more growth indication factors can be selected from concentration of the one or more identified pathogens, a variable indicating concentration of the one or more identified pathogens, or a variable that is a function of the concentration of the one or more identified pathogens.

When the tests are to be performed using an electrochemical sensor, all or a portion of the AST test samples can be suitable for use with the electrochemical sensor as an assay sample and/or the AST test samples can be combined with one or more reagents to form an AST assay sample that is suitable for use with the electrochemical sensor. Accordingly, each AST assay sample can include, consist of, or consist essentially of one of the AST test samples. The one or more AST assay samples generated from the control AST samples serve as control assay samples. The AST assay samples can be in direct physical contact with the electrochemical sensor and/or can be in direct physical contact with an electrode on the electrochemical sensor. In one example, the AST assay sample is a drop of liquid in contact with a working electrode on the electrochemical sensor.

A determination is made as to whether the one or more identified pathogens in the AST assay samples can be classified as showing growth at decision block 163. For instance, when the growth indication factors include concentration of the one or more identified pathogens, the concentrations resulting from the assay at process block 162 can be compared to the concentration of the pathogen in the control assay samples (the control concentration). When more than one control assay samples are generated, the control concentration can be generated by approaches including, but not limited to, one or more approaches selected from the group consisting of: averaging the concentrations of the identified pathogens in the control assay samples, taking the median of the concentrations of the identified pathogens in the control assay samples, taking the highest value of the concentrations of the identified pathogens in the control assay samples, or averaging the two highest values of the concentrations of the identified pathogens in the control assay samples. While the flow is disclosed in the context of directly comparing concentration values, it is not always necessary to determine and/or directly compare concentration values. For instance, other growth indication factors can be compared. As an example, the assays can include voltammetry of each AST assay sample. In these instances, the signal generated from voltammetry of each assay can indicate concentration levels and can serve as a growth indication factor. Accordingly, the voltammetry signal levels can be compared in order to effectively compare concentration levels. As an example, a voltammetry signal can be monitored for some period of time and an average of the signal for a window in that period of time can serve as the voltammetry signal level to be compared against other voltammetry signal levels. In one example, the period of time is a minute and the window is the last ten seconds of that time period. In some instances, concentration is not used to measure growth. For instance, the number of pathogens in the assay samples can be used to measure growth.

While an increased concentration of pathogen relative to the control concentration may be considered growth, in some instances, the pathogen is only considered to show growth when the level of increase in concentration is significant. For instance, the flow can be configured such that a pathogen is only considered to show growth when the concentration of pathogen in an AST assay sample relative to the control concentration shows an increase of more than 50%, 100%, or 200% and/or when a factor that indicates a concentration level or is a function of the concentration level indicates an increase of more than 50%, 100%, or 200%.

When the determination at decision block 163 is positive (classified as showing pathogen growth) for a particular AST assay sample, the pathogen is reported as being resistant to the concentration of the antimicrobial in the AST culture sample (process block 164). When the determination at decision block 163 is negative (classified as not showing pathogen growth) for a particular AST assay sample, the pathogen is reported as being susceptible to the concentration of the antimicrobial in the AST culture sample (process block 164). The results for at least each of the AST assay samples that include one or more of the identified antimicrobials can be reported.

As is illustrated by process block 164, other classifications are possible in addition to positive and negative. For instance, an AST assay sample can be classified as showing intermediate growth, resistance to an antibiotic or susceptibility to an antibiotic.

The duration of the susceptibility cultures is measured from the start of the culture at process block 152. The susceptibility culture time is considered to be zero minutes at the start of the susceptibility culture. In general, a susceptibility culture is considered to be started when the AST culture sample is heated. Accordingly, when a robotic system such as the UtiMax or BsiMax is used to conduct the susceptibility culture, the susceptibility culture is considered to be started when the UtiMax or BsiMax starts to heat the sample.

The assays (growth tests) in the antimicrobial susceptibility testing phase can be performed using the same assays as were used in the pathogen characterization phase. For instance, the growth tests in the antimicrobial susceptibility testing phase be performed manually or using a robotic system such as the UtiMax or BsiMax from GeneFluidics, Inc. of Irwindale, Calif., USA. Additionally, the reagents used in these assays can be purchased in reagent kits such as the UtiMax and the BsiMax reagent kit from GeneFluidics, Inc. of Irwindale, Calif., USA. The “16X PID/AST Sensor Chips” are electrochemical sensors sold by GeneFluidics, Inc. of Irwindale, Calif., USA for manual or automated use with the UtiMax and the BsiMax reagent kits. As noted above, these growth tests can be performed on site.

When using a robotic system such as the UtiMax or BsiMax, the testing time period from a user receiving or system generating the AST test sample to a user receiving the results of the assay(s) (indication of the growth of pathogen in an AST assay sample) is generated is generally on the order of 180 minutes for a raw sample of urine and 180 minutes for a raw sample of whole blood. When manually performing an assay, the testing time period from a user receiving the AST test sample to a user receiving the results of the assay(s) (indication of the growth of pathogen in an AST assay sample) is generated is generally on the order of 180 minutes for urine and 180 minutes for whole blood. Accordingly, the testing time period can be greater than 20 minutes, minutes or 120 minutes and/or less than 180 minutes, 240 minutes or 360 minutes.

The results of the Antimicrobial Susceptibility Testing (AST) phase are available in a time period that is approximately equal to the sum of the time to prepare the AST culture samples, the AST culture time, and the testing time period. Accordingly, the results of the antimicrobial susceptibility testing phase can be received in a time period greater than 30 minutes, 60 minutes and/or less than 5 hours or 3 hours from a user of a robotic system or the robotic system receiving the one or more test samples from which the Initial AST sample(s) is prepared.

A prescription can be made using one, two, or three factors selected from the group consisting the pathogen identity, pathogen concentration in the raw sample, antimicrobial resistance, or antimicrobial susceptibility. In an example where all three factors are used, guidelines that associate one or more particular antimicrobial medications with particular pathogens can be used to identify one or more antimicrobials that can be used to treat the identified pathogen(s). The one or more identified antimicrobials that are reported as susceptible can then be selected for a prescription. This prescription process avoids the prescription of antimicrobials to which the identified pathogen is resistant and accordingly reduces the creation of additional resistance. Additionally, using the concentration of pathogen in the raw sample as a factor in determining dosage can reduce overdosing and/or underdosing and can further reduces the creation of additional resistance.

### EXAMPLE 1

In an example of the pathogen characterization phase, a sample of 1 mL of whole blood is collected in a BD vacutainer tube from a patient with suspected bacteremia. This tube is placed in the GeneFluidics BsiMax system within 15 minutes of sample collection. The system adds 2 mL of RBC lysis buffer to the sample, then incubates this sample at room temperature for 9 minutes. Then, the system centrifuges the tube for 5 minutes at high speed to concentrate any potential bacterial pathogens along with the majority of red blood cells and red blood cell debris. The system then removes 2.5 ml of supernatant from the tube and places it in the waste compartment of the system. To the remaining pellet in the tube, the system adds 2 mL of RBC lysis buffer and incubates for 9 minutes at room temperature.

At the conclusion of this incubation, the system centrifuges the tube again for 5 minutes at high speed. The system then removes 2 mL of supernatant and disposes of it in the waste compartment. The system adds 2 mL of Mueller Hinton broth to the remaining pellet at the bottom of the tube and mixes thoroughly to ensure that the viable bacteria are suspended in the culture sample. The system incubates this sample at 37 C for 5 hours. At the conclusion of the viability culture, the system centrifuges the tube for 5 minutes at high speed. The system then removes 2.8 mL of supernatant and discards it. The system then mixes the remaining pellet at the bottom of the tube and removes 50 uL, placing it in a separate tube containing 1 mL of Mueller Hinton broth. This separate culture sample will be used for AST testing if the original sample tests positive.

To the remaining 150 uL in the original tube, the system adds 90 uL of 1M NaOH and incubates at room temperature for 5 minutes. The system then adds 60 uL of 1M HCl to the same tube. Immediately after adding HCl to the sample, the system delivers 10 uL of lysed sample to each of 14 sensors functionalized with different DNA probes to hybridize with the 16S rRNA of various bacteria species or groups (E. coli, P. aeruginosa, Enterobacteriaceae, etc.). The chip on which these sensors are contained is incubated at 43 C for 30 minutes to facilitate hybridization of the probes with the rRNA of any samples present. Following the hybridization step, the system washes the chip with deionized water and dries the chip with compressed air. The system then adds 10 uL of horseradish peroxidase in 1% casein onto each of the 16 sensors of the chip and incubates at room temperature for 5 minutes. The system then washes and dries the chip again. Following this final wash step, the system adds 40 uL of TMB-hydrogen peroxide solution to each of the 16 sensors of the chip. The system then pauses 30 seconds to allow the reaction on the sensor to reach equilibrium, and then uses an integrated reader to make contact with the electrodes of the chip, apply a voltage, and then measure the resulting current for 1 minute. The average current recorded over the last 10 seconds of this reading comprises the signal used for analysis. Using predetermined data, the Limit of Blank (LOB) is determined to be 25 nA and can determined by testing contrived negative samples of blood and then generating a cutoff value to distinguish positive and negative samples as defined in the CLSI guideline documents. For example, 20 negative samples can be tested and the LoB determined to be the second highest signal generated by those 20 samples. In this case, each sensor type would have its own established LoB but the EC sensor is the only sensor type generating a signal that exceeds this predetermined value. The signal measured in this experiment for the sensor functionalized with E. coli DNA probes produces a signal of 100 nA. All other sensors give a reading less than 25 nA. Because the signal from the sensor corresponding to E. coli is the only one giving a signal in excess of the LoB signal, the sample is considered positive for E. coli and negative for all other targeted pathogens. This assay output indicates that the concentration of E. coli in the original sample exceeds the limit of detection, which was found to be 1 CFU/mL in validation testing.

### EXAMPLE 2

A sample of 1 mL of urine is collected in a BD vacutainer tube from a patient with suspected urinary tract infection. This tube is placed in the GeneFluidics UtiMax system within 15 minutes of sample collection. Then, the system centrifuges the tube for 5 minutes at high speed to concentrate any potential bacterial pathogens. The system then removes 0.8 ml of supernatant from the tube and places it in the waste compartment of the system. At the conclusion of this incubation, the system centrifuges the tube again for 5 minutes at high speed. The system then removes 1.8 mL of supernatant and discards it in the waste compartment. The system then mixes the remaining pellet at the bottom of the tube and removes 50 uL, placing it in a separate tube containing 1 mL of Mueller Hinton broth. This separate culture sample will be used for AST testing if the original sample tests positive.

To the remaining 150 uL in the original tube, the system adds 90 uL of 1M NaOH and incubates at room temperature for 5 minutes. The system then adds 60 uL of 1M HCl to the same tube. Immediately after adding HCl to the sample, the system delivers 10 uL of lysed sample to each of 14 sensors functionalized with different DNA probes to hybridize with the 16S rRNA of various bacteria species or groups (E. coli, P. aeruginosa, Enterobacteriaceae, etc.). The chip on which these sensors are contained is incubated at 43 C for 30 minutes to facilitate hybridization of the probes with the rRNA of any samples present. Following the hybridization step, the system washes the chip with deionized water and dries the chip with compressed air. The system then adds 10 uL of horseradish peroxidase in 1% casein onto each of the 16 sensors of the chip and incubates at room temperature for 5 minutes. The system then washes and dries the chip again. Following this final wash step, the system adds 40 uL of TMB-hydrogen peroxide solution to each of the 16 sensors of the chip. The system then pauses 30 seconds to allow the reaction on the sensor to reach equilibrium, and then uses an integrated reader to make contact with the electrodes of the chip, apply a voltage, and then measure the resulting current for 1 minute. The average current recorded over the last 10 seconds of this reading comprises the signal used for analysis. Using predetermined data, the LoB is determined to be 25 nA. The signal measured in this experiment for the sensor functionalized with E. coli DNA probes produces a signal of 100 nA. All other sensors give a reading less than 25 nA. Because the signal from the sensor corresponding to E. coli is the only one giving a signal in excess of the LoB signal, the sample is considered positive for E. coli and negative for all other targeted pathogens.

### EXAMPLE 3

In an antimicrobial susceptibility testing phase, a local antibiogram can be used to identify antibiotics for treating the pathogen identified in Example 1. In one example, the identified antibiotics could be ciprofloxacin and imipenem. Susceptibility cultures can be started. For instance, a susceptibility culture including ciprofloxacin can be generated by diluting a portion of the raw sample to 5×10⁵ CFU/mL and distributing the result into a vial with 100 μL of culture media (Mueller-Hinton broth) and 0.4 μg of a lyophilized version of the ciprofloxacin. A second susceptibility culture including imipenem can be generated by diluting a portion of the raw sample to 5×10⁵ CFU/mL and distributing the result into a vial with a 0.4 μg of a lyophilized version of the imipenem and 100 μL of culture media (Mueller-Hinton broth).

The imipenem susceptibility test sample can be used to generate an imipenem test sample shortly after the imipenem susceptibility culture was started. The Proteus automated system can begin to prepare a test sample from the imipenem susceptibility test sample by transferring 30 μL of the imipenem susceptibility test sample to a microcentrifuge vial in the machine. Then, the Proteus automated system can add 12 μL of 1M NaOH to the 30 μL sample. This sample can be incubated at room temperature for 2 minutes. The Proteus system can then add 8 μL of 1M HCl. Ten microliters of the result can be placed on each of 4 sensors of the 16 available on the GeneFluidics, Inc. 16X EC chip. This chip can be incubated in the Proteus machine for 11 minutes at 43° C. After completion of this incubation, the chip can be washed and dried by Proteus. Ten microliters of 0.5 U/mL anti-fluorescein horseradish peroxidase (HRP) Fab fragments (Roche; diluted in 1.0% casein in 1 M phosphate-buffered saline, pH 7.4) can be injected into the sensor chambers and incubated for 2 minutes at 25° C. The final reagent addition can be 40 μL of 3,3′,5,5′-tetramethylbenzidine (TMB)-H₂O₂ solution (K-Blue Low Activity TMB Substrate; Neogen, Lexington Ky.) and incubated for 30 seconds to provide the imipenem test sample.

The ciprofloxacin susceptibility test sample can be used to generate a ciprofloxacin test sample shortly after the ciprofloxacin susceptibility culture was started. The Proteus automated system can begin to prepare a test sample from the ciprofloxacin susceptibility test sample by transferring 30 μL of the ciprofloxacin susceptibility test sample to a microcentrifuge vial in the machine. Then, the Proteus automated system can add 12 μL of 1M NaOH to the 30 μL sample. This sample can be incubated at room temperature for 2 minutes. The Proteus system can then add 8 μL of 1M HCl. Ten microliters of the result can be placed on each of 4 sensors of the 16 available on the GeneFluidics, Inc. 16X EC chip. This chip can be incubated in the Proteus machine for 11 minutes at 43° C. After completion of this incubation, the chip can be washed and dried by Proteus. Ten microliters of 0.5 U/mL anti-fluorescein horseradish peroxidase (HRP) Fab fragments (Roche; diluted in 1.0% casein in 1 M phosphate-buffered saline, pH 7.4) can be injected into the sensor chambers and incubated for 2 minutes at 25° C. The final reagent addition can be 40 μL of 3,3′,5,5′-tetramethylbenzidine (TMB)-H₂O₂ solution (K-Blue Low Activity TMB Substrate; Neogen, Lexington Ky.) and incubated for 30 seconds to provide the ciprofloxacin test sample.

The test samples were tested for concentration using a test that served as the base susceptibility test or the zero susceptibility culture time test. For instance, a Helios potentiostat from GeneFluidics, Inc. was connected to the electrodes of the sensor chips and a potential of −250 mV was applied across the electrodes for one minute. During this time, the current generated by the electrochemical reaction was measured by the potentiostat. The average current measured in the final 10 seconds of the potentiostat reading constituted the final result of the assay. The average current signal of the four sensors reporting the assay results was 30 nA for the imipenem susceptibility test sample and 30 nA for the ciprofloxacin susceptibility test sample. The current levels were respectively considered to represent the concentration for the imipenem base susceptibility test and for the ciprofloxacin susceptibility base test.

The imipenem susceptibility culture and the ciprofloxacin susceptibility culture were each tested according to a susceptibility-testing schedule that had susceptibility testing time appointments at 0 minutes, 60 minutes, 90 minutes, and 120 minutes. The imipenem base susceptibility test and the ciprofloxacin susceptibility base test described above served as the test for the 0 minute susceptibility testing time appointments. The process that was used to prepare the test samples for the base susceptibility tests was to be used to generate test samples from each of the susceptibility cultures at 60 minutes, and 90 minutes. Each of the test samples were tested as described above.

The test of the 60 minute imipenem test sample provided a current of 180 nA. This result showed an increase of 500% over the current level from imipenem base susceptibility test. Accordingly, this was classified as showing growth of the E. coli. In the presence of the imipenem. As a result, the result was reported as resistant and the imipenem susceptibility culture was stopped.

The test of the 60 minute ciprofloxacin test sample provided a current of 30 nA. This result showed an increase of 0% over the current level from ciprofloxacin base susceptibility test. Accordingly, this test sample was classified as showing no growth of the E. coli. As a result, a ciprofloxacin test sample was also prepared at 90 minutes of ciprofloxacin susceptibility culture time. The 90 minute ciprofloxacin test sample was tested and produced a current level that increased 0% over the current level from ciprofloxacin base susceptibility test. Accordingly, this test sample was also classified as showing no growth of the E. coli. and the result was reported as susceptible to the concentration of ciprofloxacin in the ciprofloxacin culture and the ciprofloxacin susceptibility culture was stopped.

It is believed that improvements in culture and/or assay technologies will reduce many of the times disclosed above. Accordingly, it is believed that the time needed to prescribe antimicrobials using the pathogen identity and antimicrobial susceptibility data will decrease with time. The decrease in these times will further validate the process flows set forth above.

In many of the above discussions, an assay of a sample that was processed, and may or may not have been cultured, provides the concentration of a pathogen in the sample rather than the concentration of the pathogen in the patient. In instances where it is desirable to know and/or use the concentration of the pathogen in a patient, the concentration that results from the test or assay can be converted to concentration in the patient using calibration data. Examples of calibration data include data that associate concentration in the patient with the concentration results provided by an assay at multiple different culture times. The concentration that resulted from the assay and the culture time that was used can be compared to the to calibration data include data so as to determine the concentration in the patient or in the raw data.

The above methods and process flows often refer to a result and/or data being reported. The reporting of data and/or results can be done to one or more entities selected from a group consisting of the patient, a physician, nurse, technician, pharmacist, person involved in treatment of the patient, or electronic device involved in the treatment of the patient. When robotic systems are used to execute the assays disclosed above, it is possible for these robotic systems to be operated by technicians or even the patient. For instance, when the dosage-screening phase does not involve culturing a raw sample, the robotic system can receive the raw sample directly from the patient or a technician, can process the sample, and provide the results to the patient or a technician. As a result, the robotic systems can be located in sites that typically include pharmacies such as CVS or Walgreens. These pharmacies can have technicians that operate the robotic systems.

The above methods and flows can be used in conjunction with raw samples such as whole blood, urine, saliva, sputum, cerebrospinal fluid (CSF), and fluid collected from bronchoalveolar lavage. Examples of pathogens that can be used with these methods and process flows include, but are not limited to, E. coli, P. aeruginosa, and S. aureus. Examples of antimicrobials that can be used with these methods and process flows include, but are not limited to, antibiotics, antivirals, and antifungals.

The above description discloses a second sample generated from a first sample. For instance, the above description discloses a primary treated sample generated from all or a portion of the raw sample. However, additional processing of the first sample can occur before generation of the second sample. For instance, generating the second sample from the first sample can include using the first sample to generate an auxiliary sample followed by using the auxiliary sample to generate the second sample.

The above methods, process flows, and examples can be performed in a sequence other than the described sequence. Additionally or alternately, a portion of the steps, operations, flow blocks are optional. As an example, in some instances, raw samples such as urine can provide the desired LOD levels without culturing. Accordingly, process block 108 and the associated disclosures can be optional for certain raw samples. As an example, the decision block 118 and decision block 166 are optional. For instance, when the determination at decision block 108 is negative, the process flow can proceed directly to determination block 120. When the determination at decision block 163 is negative, the process flow can proceed directly to determination block 168.

Other embodiments, combinations and modifications of this invention will occur readily to those of ordinary skill in the art in view of these teachings. Therefore, this invention is to be limited only by the following claims, which include all such embodiments and modifications when viewed in conjunction with the above specification and accompanying drawings.

1. A method of data collection, comprising:
performing a viability culture in a sample that was generated from a raw sample such that the sample includes a pathogen when the raw sample included the pathogen, the raw sample being taken from a natural source of a pathogen; and
assaying an assay sample that was generated from an aliquoted portion of the sample such that the assay sample includes the pathogen when the sample included the pathogen,
the sample being cultured for a time period that is less than 8 and greater than or equal to 0.0 hours before the aliquoted portion of the sample was taken from the sample, and
the assay sample being assayed so as to determine whether the pathogen is present in the assay sample.

2. The method of claim 1, wherein the assay is the first assay that is performed on all or a portion of the raw sample such that the assay indicates whether the pathogen is present in the raw sample.

3. The method of claim 1, wherein the raw sample is whole blood and the sample was cultured for more than 10 minutes and less than 4 hours before the aliquoted portion of the sample was taken from the sample.

4. The method of claim 3, wherein the sample was prepared such that a Limit Of Detection (LOD) for the assay is less than 100 CF/mL.

5. The method of claim 1, wherein the raw sample is urine, a Limit Of Detection (LOD) for the assay is less than 30,000 CFU/mL, and the sample was cultured for less than 100 minutes and more than 0.0 minutes before the aliquoted portion of the sample was taken from the sample.

6. The method of claim 5, wherein the sample was prepared such that a Limit Of Detection (LOD) for the assay is less than 10,000 CFU/mL.

7. The method of claim 1, further comprising:
generating a primary treated sample before the sample is generated,
the primary treated sample being generated such that the primary treated sample includes the pathogen when the raw sample included the pathogen, and
generating the primary treated sample includes performing a purifying operation that reduce the concentration of at least one type of cell that is not the pathogen.

8. The method of claim 7, wherein performing the purifying operation includes lysing all or a portion of the raw sample.

9. The method of claim 1, further comprising:
generating a secondary treated sample before the sample is generated,
the secondary treated sample being generated such that the secondary treated sample includes the pathogen when the raw sample included the pathogen, and
generating the secondary treated sample includes performing a concentration operation on a primary treated sample such that a concentration of the pathogen in the secondary treated sample is higher than the concentration of the pathogen in the primary treated sample.

10. The method of claim 9, wherein performing the concentration operation includes centrifuging the primary treated sample.

11. The method of claim 10, wherein at least a portion of a supernatant from the centrifuge is separated from a centrifuge pellet and the secondary treated sample includes at least a portion of the centrifuge pellet.

12. The method of claim 9, wherein all or a portion of the raw sample is the primary treated sample.

13. The method of claim 9, further comprising:
generating the primary treated sample before the secondary treated sample is generated,
the primary treated sample being generated such that the primary treated sample includes the pathogen when the raw sample included the pathogen, and
generating the primary treated sample includes performing a purifying operation that reduces the concentration of at least one type of cell that is not the pathogen.

14. The method of claim 13, wherein performing the purifying operation includes lysing all or a portion of the raw sample.

15. The method of claim 13, wherein generating the primary treated sample and the secondary treated sample is included in a step of a cycle that is repeated multiple times.

16. The method of claim 9, further comprising:
generating the sample from the secondary treated sample.

17. The method of claim 16, wherein generating the sample includes adding one or more culture media to the secondary treated sample.

18. The method of claim 1, further comprising:
generating a preliminary test sample from the aliquoted portion of the sample, generating the preliminary test sample includes performing a concentration operation on the aliquoted portion of the sample such that a concentration of the pathogen in the preliminary test sample is higher than the concentration of the pathogen in the sample,
the assay sample being generated from the preliminary test sample.

19. The method of claim 18, further comprising:
generating the assay sample from the preliminary test sample.

20. The method of claim 19, wherein assaying the assay sample indicates that the pathogen is present in the assay sample, and further comprising:
performing an antimicrobial susceptibility testing phase where the susceptibility of the pathogen to one or more antimicrobials is tested,
performing the antimicrobial susceptibility testing phase including testing an AST test sample for growth of the pathogen, and
the AST sample being generated from the preliminary test sample.

21. The method of claim 1, further comprising:
generating a test sample after taking the aliquoted portion of the sample from the sample and before assaying the assay sample,
the test sample being generated such that the test sample includes the pathogen when the raw sample included the pathogen,
generating the test sample includes performing a release operation that releases the pathogen from cells that were present in the raw sample, and
the assay sample being generated from one or more of the test samples.

22. A method of data collection, comprising:
culturing a viability sample that was generated from a raw sample taken from a natural source of pathogens;
assaying an assay sample for the presence or absence of a pathogen in the raw sample;
testing an AST test sample for growth of the pathogen in the presence of an antimicrobial; and
the AST sample and the assay sample each being generated from the viability sample.

23. The method of claim 22, further comprising
generating a preliminary test sample from the viability sample after the culturing of the viability sample, and
the AST sample and the assay sample each being generated from the preliminary test sample.

24. The method of claim 22, wherein generating the preliminary test sample includes performing a concentration operation on all or a portion of the viability sample after the culturing of the viability sample, the pathogen concentration providing a preliminary test sample having the pathogen at a higher concentration than the pathogen concentration in the viability sample.

25. The method of claim 24, wherein the concentration operation includes centrifugal separation.
